(19) United States
(12) Patent Application Publication
(10) Pub. No.: US 2005/0124935 A1
(43) Pub. Date: Jun. 9, 2005

(54) Venting adapter for feeding device

(75) Inventor: Donald J. McMichael, South Jordan, UT (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/729,302

(22) Filed: Dec. 5, 2003

(51) Int. Cl. A61M 1/00

(52) U.S. Cl. 604/129

(57) ABSTRACT

The present invention is directed generally to an adapter for use with enteral tubes or catheters, and more particularly to a product that enables the venting or decompression of enteric gases through an enteral catheter. The adapter is configured to allow continuous venting with reduced concern about liquid leakage or the need for the user to limit their activities or assume certain positions during venting.

### BACKGROUND

Numerous situations exist in which a body cavity needs to be catheterized to achieve a desired medical goal. One relatively common situation is to provide nutritional solutions or medicines directly into the stomach or intestines. A stoma is formed in the stomach or intestinal wall and a catheter is placed through the stoma. Feeding solutions can be injected through the catheter to provide nutrients directly to the stomach or intestines (known as enteral feeding). A variety of different catheters intended for enteral feeding have been developed over the years, including some having a “low profile” relative to the patient during use and those having the more traditional or non-low profile configuration.

As indicated above, there are a variety of instances in which it may be necessary to use a catheter, one of which is the not uncommon reaction following major surgery in which a patient's stomach function is impaired for a period of time. In spite of the need to supply or supplement the body with a certain level of nutrients and the like following surgery as well as in other instances of impaired or limited gastric functionality, an unfed gut can become a source of bacteria that gets into the bloodstream. These types of problems may be resolved by the introduction of nutrients through an enteral feeding device tube properly inserted through the patient's abdominal wall, gastric wall, pylorus, duodenum, and/or into the jejunum beyond the Ligament of Treitz.

A problem universal to low profile and non-low profile devices is difficulty in decompressing or venting gases from a patient thereby enabling the release of enteric gases and resulting pressure. Just as with a patient who consumes or takes in food or medication orally without the need for enteral feeding via a catheter, a patient receiving nutrition or medication through an enteral catheter may generate gases within his enteral region either as part of the digestive process or as a result of a reaction to the medication. While some level of gas and pressure is natural in either case, a higher level of gas and pressure may develop.

Another condition where enteral catheterization and feeding may be needed is if the patient cannot swallow. The inability to swallow or otherwise satisfactorily control one's neck muscles could inhibit one's ability to relieve some of the pressure and gases which build up in the enteral region. Specifically, one may not be able to vent or expel gases naturally (e.g., belching).

Alternatively, due to a patient's condition, the patient may not be able to take a medication to reduce the gases and relieve the pressure. Furthermore, the addition of nutritional fluids or medications through a catheter that does not provide venting will result in a pressure increase inasmuch as more fluids are now present in the same area than immediately before the addition. While some of these fluids may be absorbed into the body over a period of time, the resulting pressure may be uncomfortable until some of the fluids are absorbed into the body.

In the past, an extension set was intermittently attached to the enteral feeding device to allow venting of the gases and pressures discussed above. However, that method of venting has several drawbacks. First, an extension set used to provide liquid nutritional supplement and/or medication to the enteral feeding device does not prevent liquids from passing or escaping therethrough. Although it may be possible to reduce liquid escape or back flow with the prior technique, additional effort is required to make sure that the end of the extension set is maintained at a sufficient height and that the user activity is minimized or restricted during the venting process. Second, as liquids can escape through prior catheters and extension sets and because activity of the user may need to be limited to maintain the elevation of the extension set above the enteral feeding device during the venting process, the prior techniques for venting are performed on an intermittent basis. Regardless of a patient's ability to perform the venting on their own, intermittent venting can create difficulties. For example, if not done on a regular basis it can result in discomfort to the user. However, if done on a regular basis, it may inconvenience a patient to minimize or restrict their activities and/or to need to assume certain positions several times a day to enable or facilitate venting. Furthermore, if the patient is not able to perform the necessary venting on their own, the assistance of a clinician to perform the venting may be required multiple times a day.

Accordingly, while a number of improvements have been made to conventional enteral tubes, there remains the need and the desire for a way to vent enteral feeding devices not only on a continuous basis, but such that liquids do not escape during the venting procedure, as well as the need for a venting mechanism and procedure which do not restrict the activity or position of a user while the venting is being performed. The present invention meets these needs.

### SUMMARY OF THE INVENTION

In response to the difficulties and problems discussed above an adapter configured for use with a feeding tube has been developed. More specifically, one aspect of this invention is directed to a venting adapter having two openings in fluid communication with one another, and an insert comprising at least in part a porous material. One of the openings of the adapter is capable of insertion into the feeding tube. The second opening of the adapter may be configured to receive the insert; the insert being positioned within the second opening. The adapter may be configured so as to allow gases but not liquids to pass therethrough. The adapter may also incorporate a mechanism adapted to removably secure the adapter to a feeding tube.

Another embodiment of the present invention is directed to an adapter adapted for use with an enteral feeding device feeding tube. The adapter may generally include a first opening and a second opening in fluid communication, a male end and an insert being at least in part a porous material. The first opening is located in the male end and is adapted for insertion into a port of the feeding tube. The second opening is configured to receive the insert which may be positioned between the first opening and the second opening. The insert is configured so as to allow gases but not liquids to pass therethrough.

The present invention is also directed to an apparatus which enables enteral feeding as well as the ability to vent an enteral cavity. One embodiment may include an enteral feeding device and an adapter configured for connection to the feeding device so as to allow gases but not liquids to vent therethrough. More specifically, an embodiment may include an adapter having a first opening and a second opening in fluid communication, a male end, and an insert including comprising at least in part a porous material. The first opening may be located in the male end of the adapter and should be adapted for insertion into a port of a feeding device. The second opening may be configured to receive the insert which is positioned near second opening. The adapter may be configured so as to allow gases but not liquids to vent therethrough.

Yet another aspect of the present invention is directed to a venting adapter configured for use with a feeding tube. The adapter may generally include two openings in fluid communication with one another, and an insert comprising at least in part a porous material. One of the openings being capable of insertion into the feeding tube and the second opening configured to receive the insert which is positioned about the second opening. The adapter being configured so as to allow gases but not liquids to pass therethrough when the insert is positioned about the second opening.

Another aspect of the present invention is directed to a venting adapter configured for use with a feeding tube. The adapter including two openings in fluid communication with one another, and an insert comprising at least in part a porous material. One of the openings is capable of insertion into the feeding tube. The insert of the adapter being positioned between the first opening and the second opening and wherein the adapter is configured to allow gases but not liquids to pass therethrough when the insert is positioned between the first opening and the second opening.

The invention will be more fully understood and further features and advantages will become apparent when reference is made to the following detailed description of exemplary embodiments of the invention and the accompanying drawings.

### BRIEF DESCRIPTION OF THE DRAWINGS

The above and other objects, features and advantages of the invention will become apparent from consideration of the subsequent detailed description presented in connection with the accompanying drawings in which:

FIG. 1 shows a perspective view of an adapter in accordance with the present invention;

FIG. 2 is a cross-sectional view of one embodiment of the present invention, the adapter having a porous insert positioned therein;

FIG. 3 is a cross-sectional view of one embodiment of the present invention, the adapter having a removable insert positioned therein;

FIG. 4 is a cross-sectional view of an exemplary low profile catheter suitable for receiving an adapter of the present invention; and

FIG. 5 is an illustration of an adapter in accordance with the present invention attached to an exemplary low profile catheter.

### DETAILED DESCRIPTION OF THE PRESENT INVENTION

Reference will now be made to the drawings in which the various elements of the present invention will be given numeral designations and in which the invention will be discussed so as to enable one skilled in the art to make and use the invention.

The present invention relates generally to enteral tubes or catheters, and more particularly to a product that may be used therewith which enables the venting or decompression of gases through an enteral tube.

It will be appreciated that throughout the disclosure reference is made to enteral feeding catheters for purposes of ease of reading and understanding the disclosure, however, the present invention is not intended to be limited to enteral feeding devices, enteral feeding tubes or the like.

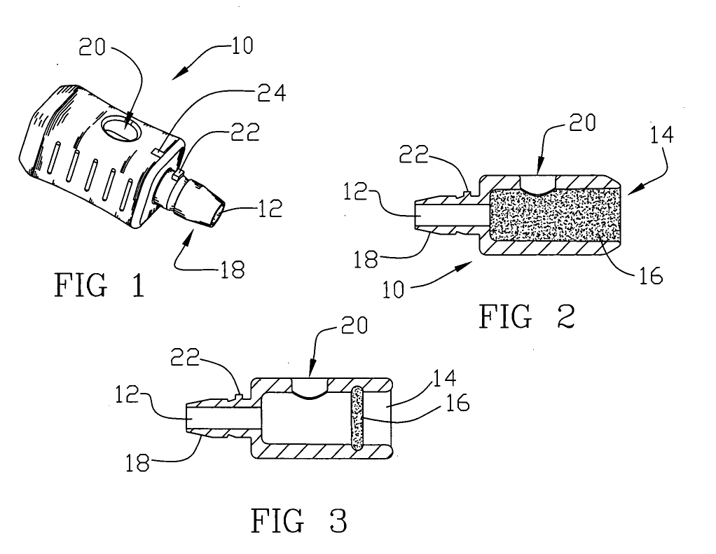

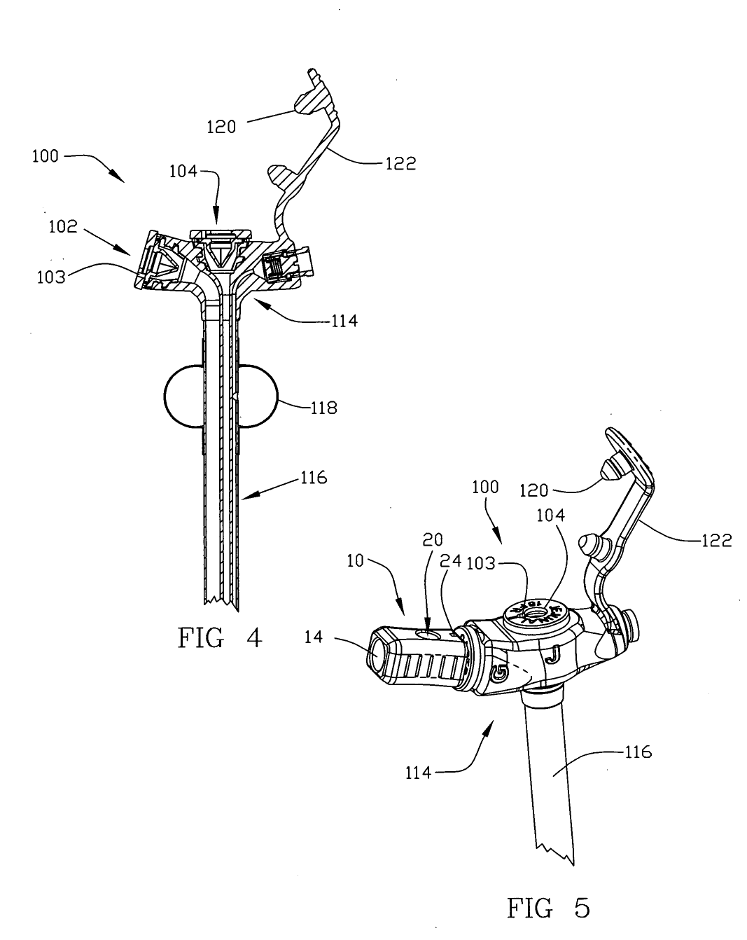

Referring now to FIGS. 1 and 2, there is shown a perspective view and a sectional view, respectively, of an adapter 10 made in accordance with the teachings of the present invention. The adapter 10 is generally configured for use with an enteral feeding catheter, such as those discussed below. The adapter 10 is shown having two openings 12, 14 (FIGS. 1-3 and FIGS. 2, 3 and 5, respectively) in fluid communication with one another, and an insert 16 (FIGS. 2 and 3) comprising at least in part a porous material. Desirably, one of the openings 12 is capable of insertion into a catheter or feeding tube 100 (FIGS. 4 and 5), and may be found in a male end 18 (FIGS. 1 and 2) of the adapter 10. It will be appreciated that the insert 16 (FIGS. 2 and 3) may be positioned within the adapter 10 in a variety of manners (some permanently and some less than permanent) including, for example, overmolding of the adapter about the insert, friction fit, the insertion of an insert and/or a rim about a portion of the insert into a groove in the adapter (e.g., groove and boss relationship), or the like to retain or assist in retaining the insert 16 (FIGS. 2 and 3) in the adapter 10. The adapter 10 may desirably allow for removal of the insert 16 so as to provide for cleaning and/or replacement. In those embodiments in which the insert 16 (FIGS. 2 and 3) is removable, the second opening 14 (FIG. 5) is desirably configured to receive the insert 16 (FIGS. 2 and 3). In use the insert 16 may be positioned between the first opening 12 (FIGS. 1, 2 and 3) and the second opening 14 (FIGS. 3 and 5), wherein the adapter 10 (FIGS. 1, 2, 3 and 5) is configured so as to allow gases but not liquids to pass therethrough. It will be appreciated that an alternative adapter (not shown) may have the insert positioned over or about the second opening yet still be able to achieve the same function. It will be appreciated that all such combinations or variations are contemplated by the present invention.

As noted above, the first opening 12 (FIGS. 1, 2 and 3) may be positioned in a male end or portion 18 (FIGS. 1, 2 and 3) of the adapter 10. The male end or portion 18 (FIGS. 1, 2 and 3) of the adapter 10 is desirably such that it is adapted or configured to open a valve (e.g., a duckbill valve) or the like in a port (e.g., the gastric port 102 (FIG. 4)) or lumen (not shown) of the catheter 100 (FIGS. 4 and 5) upon insertion thereinto.

Although the adapter may be releasably secured or connected to a catheter by friction alone, the adapter 10 may also include a mechanism adapted to releasably secure the adapter 10 to a catheter 100. Suitable mechanisms adapted to releasably secure the adapter 10 to a catheter include but are not limited to a bayonet lock, boss and detent, lock and key mechanism, snap fit, screw threads, grooves and the like. Other suitable mechanisms which are known in the art are also contemplated. It will be appreciated that in some embodiments, only part of a suitable mechanism may be present in or on the adapter 10 while another portion is present in or on the catheter 100. That is, for example, where a lock and key mechanism is used, the adapter 10 may contain the key or protrusion portion thereof, while the catheter 100 contains the lock or recess portion thereof. Of course, it will be appreciated that another embodiment of the present invention may be such that the lock or recess portion of the mechanism is contained in the adapter while the key or protrusion is present on the catheter. Again, any suitable combination and configuration thereof is contemplated by the present invention and intended to be claimed herein.

The adapter 10 of FIGS. 1, 2 and 3 is shown as having an optional protrusion or key 22 which may be received in a port 102, 104 (FIG. 4 and FIGS. 4 and 5, respectively) of the catheter 100 (FIGS. 4 and 5) and rotated such that the adapter 10 is positioned as desired (e.g. considering the position of second opening 14 (FIGS. 2 and 5) and the profile of the adapter 10 relative to the catheter 100 and patient (not shown)). It will be appreciated that a desired in use positioning of the adapter 10 relative to a catheter 100 (FIGS. 4 and 5) may be such as that shown in FIG. 5 in which the adapter 10 is not removable until the protrusion 22 (FIGS. 1-3) is aligned with the lock or recess portion 103 (FIG. 4) of the port 102 (FIG. 4). The port 102 may include a portion of a SECUR-LOK* lock and key mechanism (available from Ballard Medical Products, a subsidiary of the assignee) which is capable of receiving an adapter 10 with such a protrusion 22 (FIGS. 1-3). To facilitate attachment or detachment of the adapter 10 and catheter 100 (FIGS. 4 and 5) the adapter and/or catheter may have a lock orientation indicator 24 such as that shown in FIGS. 1 and 5. Although in most instances, the adapter 10 will generally be connected to the gastric port 102 or lumen of the catheter 100, if, for example, the catheter has another nutritional or medicinal port such as the jejunal port 104 shown in FIGS. 4 and 5, adapter 10 of the present invention could also be used therewith, although a different sized or shaped adapter may be more desirable depending on the location of the other port 104.

While it is desired that the adapter 10 (FIGS. 1-3 and 5) be removably inserted or secured to a catheter 100 (FIGS. 4 and 5), it is contemplated that because the insert 16 (FIGS. 2 and 3) is removable in one or more embodiments, the adapter 10 (FIGS. 1-3 and 5) could be more permanently affixed yet still allow the nutritional fluids, medications, or the like to be passed therethrough into the catheter 100 (FIGS. 4 and 5) while the insert 16 (FIGS. 2 and 3) is removed and then upon completion of the provision of nutritional fluids, medications, or the like the insert 16 could be repositioned in or about the second opening 14 (FIGS. 2-3 and 5) as discussed above. It will be appreciated that depending on the construction of the adapter 10 and/or the insert 16 the useful life of each may vary. Accordingly, there may be a need to replace either the adapter 10 or the insert 16 before the useful life of the other component has expired. Because the adapter 10 and/or insert 16 may be exposed to less than sterile conditions (e.g., enteral liquids and gases, even though the liquids should not pass through the insert), the adapter 10 and/or insert 16 may need to be replaced on a periodic basis (e.g., weekly, bi-weekly, etc.) depending on the amount and/or conditions of use.

Turning now to the make up of the insert, exemplarily shown as 16 in FIGS. 2 and 3. The inserts contemplated by the present invention are desirably such that will allow gases but not liquids to pass therethrough. Stated alternately, the inserts of the present invention desirably are vapor permeable and liquid impermeable. The insert 16 may comprise any suitable material or combination thereof. Exemplary suitable materials for the insert include but are not limited to reticulated polymer foams, expanded polymers (such as Porex® expanded polymers available from Porex Corporation, having offices in Fairburn, Ga.), expanded PTFE (such as Gore-Tex® expanded PTFE available from W.L. Gore & Associates, Inc., having offices in Newark, Del.), and porous metals (or powdered metals). As will be appreciated the rate at which the gases are allowed to pass through the insert 16 is not critical so long as it is sufficient to achieve the desired venting. It will also be appreciated that venting rate may be affected or controlled in part by the composition of the insert. Nevertheless, in most embodiments of an adapter of the present invention intended for use with adult catheters, it is generally desirable for the insert to be able to allow at least 3-5 liters of gas to pass therethrough per hour. For use with a pediatric catheter, it may be desirable for the insert in an appropriately sized adapter to be able to allow at least 1-2 liters of gas to pass therethrough per hour. Further, it will be appreciated that the filter or insert media may be hydrophobic or hydrophilic, although it is desired that the insert or insert media be generally hydrophobic. Where the insert is or contains a hydrophobic filter media or where the insert media is at least in part hydrophobically treated, the media may have larger pore sizes and therefore a higher flow rate therethrough (as compared to a hydrophilic or hydrophilically treated media) as the insert will be less likely to absorb liquids, become saturated and allow liquid to pass therethrough.

The present invention contemplates and is intended to include numerous other optional aspects which may be included or added to the adapters and/or catheters of the present invention. Exemplary optional aspects include, for example, a hole or well 20 (FIGS. 1-3 and 5) in the adapter 10 (FIGS. 1-3 and 5) which is configured to receive a plug, such as that which may be included with some catheters. The well 20 (FIGS. 1-3 and 5) enables a plug such as that shown at 120 in FIGS. 4 and 5 to be inserted therein and thus not be loose while the adapter 10 is attached to the catheter 100. The ability to retain the plug 120 in well 20 significantly reduces the chances that the lanyard 122 (FIGS. 4 and 5) associated with plug 120 will get caught in or tangled up with something (e.g. tubing or clothing) and thereby cause or result in the dislodgement of the catheter and/or the creation of discomfort to the patient if sufficient force is translated to the catheter. It is of note that in at least one embodiment of the present invention that the ability to vent may be temporarily suspended by the insertion of plug 120 or the like into opening 14 of the adapter. Venting can resume upon removal of the plug 120 or the like from the opening 14.

It will be appreciated that the adapter may be of any suitable shape and size, although the adapter will desirably have a low profile if used with a catheter having a low profile. The openings 12, 14 (FIGS. 1-3 and FIGS. 2, 3 and 5, respectively) may be located on any side of the adapter 10 (FIGS. 1-3 and 5) which when connected with or to a catheter 100 (FIGS. 4 and 5) will allow venting. Recognizing that the adapter may be attached and then removed (e.g., during provision of nutritional or medicinal fluids) numerous times, the second opening 14 (FIGS. 2-3, and 5) is desirably located at the end of the adapter 10 which is opposite the male end 18 (FIGS. 1 and 2) of the adapter 10. Such a location of the second opening 14 may help avoid accidental occlusion of the opening.

Referring now to FIGS. 4 and 5, there is shown a sectional view and a partial side view, respectively, of a catheter 100 made in accordance with the teachings of the present invention. The catheter 100 generally includes a head 114, a shaft 116, and a retention mechanism or expandable member, shown as balloon 118 in FIG. 4. The catheter may be of a “low profile” as shown in FIGS. 4 and 5 such that the head 114 may rest on or near the skin of a patient (not shown) when the catheter 100 is properly positioned within the patient, or the catheter may take a more traditional or non-low profile configuration (such as that discussed in U.S. Pat. No. 4,701,163 to Parks) wherein the head of the catheter generally extends away from the skin of the patient when the catheter is properly positioned in the patient and wherein the head is frequently larger as concealment or the potential therefore may not be of significant concern.

In all prior traditional or low profile catheters extension sets (not shown) were necessary to achieve the desired or necessary venting. With a significant length of tubing associated therewith, the extension set adds considerable bulk to the catheter, making concealment of the catheter and its components more difficult and troublesome. This is especially true with the use of a low profile catheter as the portion of the catheter which extends above the patient's skin is generally minimal and does not include much tubing external to the patient so as to conceal the presence of the catheter as well as to reduce the likelihood that the device will get caught on or in something and potentially dislodge the catheter from the patient. The present invention provides a solution which overcomes problems and difficulties associated with the prior catheters.

As suggested above, the adapter of the present invention is intended to work with a variety of enteral feeding devices. Exemplary enteral feeding devices include but are not limited to gastrostomy devices, jejunostomy devices, transgastric-jejunal devices and the like any of which may be low profile or non-low profile. A more detailed description and discussion of specific embodiments of suitable catheters may be found in U.S. Pat. No. 5,997,503 to Willis et al. and U.S. Pat. No. 5,997,546 to Foster et al., and commonly assigned copending U.S. patent application Ser. No. 10/159,514 (Attorney Docket No. 17,508A), filed May 31, 2002.

It should be appreciated that each example and drawing is provided by way of explaining the invention, and not as a limitation of the invention. For example, features illustrated or described with respect to one aspect may be used with another aspect to yield still a further aspect of the invention. These and other modifications and variations are within the scope and spirit of the invention.

It should further be appreciated that each aspect of the present invention may not possess each and every component described or contemplated hereby and/or may not possess each and every advantage described or contemplated herein but all such aspects are nevertheless contemplated to be within the scope of the disclosure and the attached claims.

Each of the patents, applications, and/or references mentioned, referred to, or discussed herein is herein incorporated by reference in its entirety.

While various patents and other reference materials have been incorporated herein by reference, to the extent there is any inconsistency between incorporated material and that of the written specification, the written specification shall control. In addition, while the invention has been described in detail with respect to specific aspects thereof, those skilled in the art, upon obtaining an understanding of the invention, may readily conceive of alterations to, variations of, and equivalents to the described aspects. It is intended that the present invention include such modifications and variations as come within the scope of the appended claims and their equivalents.

1. A venting adapter configured for use with a feeding tube, the adapter having two openings in fluid communication with one another, and an insert comprising at least in part a porous material; one of the openings being capable of insertion into the feeding tube; the second opening configured to receive the insert; the insert being positioned within the second opening; wherein the adapter is configured so as to allow gases but not liquids to pass therethrough.

2. The adapter of claim 1 wherein the first opening being within a male end, the male end being adapted to open a duckbill valve.

3. The adapter of claim 1 wherein the insert is removable.

4. The adapter of claim 1 further comprising a mechanism adapted to removably secure the adapter to a feeding tube.

5. The adapter of claim 4 wherein the mechanism adapted to removably secure the adapter to the feeding tube is selected from the group comprising a bayonet lock, boss and detent, lock and key mechanism, snap fit, screw threads, and grooves.

6. The adapter of claim 1 wherein at least a portion of the porous material of the insert is selected from the group consisting of reticulated polymer foams, expanded polymers, expanded PTFE, porous metals, and powdered metals.

7. The adapter of claim 1 wherein the insert is at least in part hydrophobic or hydrophobically treated.

8. An adapter adapted for use with an enteral feeding device, the adapter having a first opening and a second opening in fluid communication, a male end and an insert comprising at least in part a porous material; the first opening being in the male end and being adapted for insertion into a port of the feeding tube; the second opening configured to receive the insert; the insert being positioned between the first opening and the second opening; wherein the insert is configured so as to allow gases but not liquids to pass therethrough.

9. An apparatus which enables enteral feeding as well as the ability to vent an enteral cavity comprising:
an enteral feeding device; and
an adapter configured for connection to the feeding device so as to allow gases but not liquids to vent therethrough.

10. The apparatus of claim 9 wherein the adapter comprises a first opening and a second opening in fluid communication, a male end, and an insert comprising at least in part a porous material; the first opening being in the male end adapted for insertion into a port of the feeding device; the second opening configured to receive the insert; the insert being positioned near second opening; wherein the insert is configured so as to allow gases but not liquids to pass therethrough.

11. The apparatus of claim 9 wherein the enteral feeding device is a gastronomy device.

12. The apparatus of claim 9 wherein the enteral feeding device is a jejunal feeding device.

13. The apparatus of claim 9 wherein the enteral feeding device is a transgastricjejunal device.

14. The apparatus of claim 9 wherein the enteral feeding device is a low profile device.

15. The apparatus of claim 10 wherein the insert is removable.

16. The apparatus of claim 9 further comprising a mechanism adapted to removably secure the adapter to a feeding tube.

17. The apparatus of claim 16 wherein the mechanism adapted to removably secure the adapter to the feeding tube is selected from the group comprising a bayonet lock, boss and detent, snap fit, screw threads, and grooves.

18. The apparatus of claim 10 wherein at least a portion of the porous material of the insert is selected from the group consisting of consisting of reticulated polymer foams, expanded polymers, expanded PTFE, porous metals, and powdered metals.

19. The adapter of claim 10 wherein the insert is at least in part hydrophobic or hydrophobically treated.

20. A venting adapter configured for use with a feeding tube, the adapter having two openings in fluid communication with one another, and an insert comprising at least in part a porous material; one of the openings being capable of insertion into the feeding tube; the second opening configured to receive the insert; the insert being positioned about the second opening; wherein the adapter is configured so as to only allow gases to pass therethrough when the insert is positioned about the second opening.

21. A venting adapter configured for use with a feeding tube, the adapter having two openings in fluid communication with one another, and an insert comprising at least in part a porous material; one of the openings being capable of insertion into the feeding tube; the insert being positioned between the first opening and the second opening; wherein the adapter is configured so as to allow gases but not liquids to pass therethrough when the insert is positioned between the first opening and the second opening.
